(19) United States
(12) Patent Application Publication
(10) Pub. No.: US 2024/0134068 A1
(43) Pub. Date: Apr. 25, 2024

(54) RADIATION MEASUREMENT DEVICES

(71) Applicant: SHANGHAI UNITED IMAGING HEALTHCARE CO., LTD., Shanghai (CN)

(72) Inventors: Qiusheng WU, Shanghai (CN); Yifeng JIANG, Shanghai (CN); Yangyang LIN, Shanghai (CN)

(73) Assignee: SHANGHAI UNITED IMAGING HEALTHCARE CO., LTD., Shanghai (CN)

(21) Appl. No.: 18/398,102

(22) Filed: Dec. 27, 2023

Related U.S. Application Data
(63) Continuation of application No. PCT/CN2022/103105, filed on Jun. 30, 2022.

(30) Foreign Application Priority Data
Jun. 30, 2021 (CN) 202110743349.4

(51) Int. Cl. G01T 1/16

(52) U.S. Cl. CPC G01T 1/1603

(57) ABSTRACT

A radiation measurement device may be provided. The radiation measurement device may comprise a scattering component and a first detector. The scattering component may be located between a radiation source of an imaging device and the first detector, and configured to scatter first radiation rays emitted by the radiation source into scattering rays. The first detector may be configured to collect first measurement data by detecting at least a portion of the scattering rays, the first measurement data reflecting an operation status of the radiation source.

### CROSS-REFERENCE TO RELATED APPLICATIONS

This application is a continuation of International Application No. PCT/CN2022/103105, filed on Jun. 30, 2022, which claims priority to Chinese Application No. 202110743349.4, filed on Jun. 30, 2021, the entire contents of each of which are hereby incorporated by reference.

### TECHNICAL FIELD

The present disclosure relates to X-ray imaging, and in particular, to radiation measurement devices used in X-ray imaging.

### BACKGROUND

X-ray imaging is widely used to scan a subject to generate an image of the subject. During the scan of the subject using an X-ray imaging device, due to changes of the scanning environment, hardware defects, etc., there may be an error between a desired radiation intensity and a true radiation intensity of the X-ray imaging device, which can affect the quality of the image of the subject. In addition, during the scan, a focal point of a radiation source may move due to a thermal expansion of the radiation source, which may cause artifacts in the image of the subject.

Thus, it is desired to provide a radiation measurement device capable of collecting measurement data reflecting an operation status of the radiation source.

### SUMMARY

According to an aspect of the present disclosure, a radiation measurement device may be provided. The radiation measurement device may comprise a scattering component and a first detector. The scattering component may be located between a radiation source of an imaging device and the first detector, and configured to scatter first radiation rays emitted by the radiation source into scattering rays. The first detector may be configured to collect first measurement data by detecting at least a portion of the scattering rays, the first measurement data reflecting an operation status of the radiation source.

In some embodiments, the scattering component may be further configured to transmit the first radiation rays into transmission rays. The first detector may be further configured to collect second measurement data by detecting at least a portion of the transmission rays, the second measurement data reflecting the operation status of the radiation source.

In some embodiments, the first detector may include one or more first detecting units configured to collect the first measurement data and one or more second detecting units configured to collect the second measurement data.

In some embodiments, the one or more first detecting units and the one or more second detecting units may be spaced apart from each other.

In some embodiments, the radiation measurement device may further include a processing device configured to determine, based on the second measurement data, a focal point of the radiation source.

In some embodiments, to determine a focal point of the radiation source based on the second measurement data, the processing device may be configured to determine an intensity center of the at least a portion of the transmission rays detected by the first detector based on the second measurement data. The processing device may be also configured to obtain a first relationship between the intensity center of the at least a portion of the transmission rays and the focal point of the radiation source. The processing device may be further configured to determine the focal point of the radiation source based on the intensity center of the at least a portion of the transmission rays and the first relationship.

In some embodiments, the radiation measurement device may further include an attenuation component disposed between the scattering component and the first detector, and the attenuation component may be configured to attenuate the transmission rays transmitted from the scattering component.

In some embodiments, the radiation source may be further configured to scan a subject by emitting second radiation rays toward the subject. The first measurement data may include an intensity of the at least a portion of the scattering rays detected by the first detector. The radiation measurement device may further include a processing device configured to determine an intensity of the second radiation rays based on the intensity of the at least a portion of the scattering rays detected by the first detector.

In some embodiments, to determine an intensity of the second radiation rays based on the intensity of the at least a portion of the scattering rays detected by the first detector, the processing device may be configured to obtain a second relationship between the intensity of the at least a portion of the scattering rays detected by the first detector and the intensity of the second radiation rays. The processing device may be aslo configured to determine the intensity of the second radiation rays based on the second relationship and the intensity of the at least a portion of the scattering rays detected by the first detector.

In some embodiments, the processing device may be further configured to obtain scan data of the subject collected by a second detector during the scan of the subject, and determine an intensity of radiation rays absorbed by the subject during the scan based on the scan data and the intensity of the second radiation rays.

In some embodiments, the imaging device may further include a filter located between the radiation source and the second detector, and configured to filter the second radiation rays. The scattering component and the filter may include the same material.

In some embodiments, the radiation source may be further configured to scan a subject by emitting second radiation rays toward the subject. The imaging device may further include a second detector configured to detect the second radiation rays after passing though the subject. The scattering component may be configured such that an intensity of the at least a portion of the scattering rays detected by the first detector is substantially similar to an intensity of the second radiation rays detected by the second detector.

In some embodiments, the radiation measurement device may further include a blocking component disposed between the scattering component and the radiation source. The blocking component may include a through hole for the first radiation rays to pass through to irradiate the scattering component.

According to another aspect of the present disclosure, a radiation measurement device may be provided. The radiation measurement device may comprise a scattering component and a first detector. The scattering component may be located between a radiation source of an imaging device and the first detector, and configured to transmit first radiation rays emitted by the radiation source into transmission rays. The first detector may be configured to collect first measurement data by detecting at least a portion of the transmission rays, the first measurement data reflecting an operation status of the radiation source.

According to yet another aspect of the present disclosure, an imaging system may be provided. The imaging system may comprise a radiation source, a radiation measurement device, and a processing device. The radiation source may be configured to emit first radiation rays toward the radiation measurement device and second radiation rays toward a subject. The radiation measurement device may include a first detector and a scattering component located between the radiation source and the first detector. The scattering component may be configured to scatter the first radiation rays into scattering rays, and the first detector may be configured to collect first measurement data by detecting at least a portion of the scattering rays. The processing device may be configured to determine feature information of the second radiation rays based on the first measurement data.

In some embodiments, the radiation measurement device may be located outside a tube port of the radiation source.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a block diagram illustrating an exemplary imaging system according to some embodiments of the present disclosure;

FIG. 2 illustrates an exemplary imaging system according to some embodiments of the present disclosure;

FIG. 3A illustrates an exemplary radiation measurement device according to some embodiments of the present disclosure;

FIG. 3B illustrates a top view of a reference detector according to some embodiments of the present disclosure;

FIGS. 4A-4D illustrates top views of exemplary reference detectors according to some embodiments of the present disclosure;

FIG. 5 is a schematic illustrating an exemplary radiation measurement device according to some embodiments of the present disclosure;

FIG. 6 is a schematic illustrating an exemplary radiation measurement device according to some embodiments of the present disclosure;

FIG. 7 is a schematic illustrating an exemplary radiation measurement device according to some embodiments of the present disclosure;

FIG. 8 is a block diagram illustrating an exemplary processing device according to some embodiments of the present disclosure;

FIG. 9 is a flowchart illustrating an exemplary process for determining a focal point of a radiation source of an imaging device according to some embodiments of the present disclosure; and

FIG. 10 is a flowchart illustrating an exemplary process for determining an intensity of second radiation rays according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items.

It will be understood that, although the terms “first,” “second,” “third,” “fourth,” etc., may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Conventionally, a radiation measurement device is usually arranged near a tube port of a radiation source to measure an intensity of radiation rays emitted by the radiation source, in order to prevent the radiation measurement device from being blocked by a subject during the scan of the subject. Typically, the radiation measurement device includes a flitter and a reference detector. The flitter is configured to filter radiation rays emitted by the radiation source toward the radiation measurement device. The reference detector is configured to collect measurement data by detecting radiation rays passing through the flitter, and the measurement data is used to determine the intensity of radiation rays emitted by the radiation source. However, in the scanning process, since a distance between the radiation measurement device and the radiation source is much smaller than a distance between the radiation source and an imaging detector configured to collect scan data of a subject, a radiation intensity detected by the radiation measurement device is much greater than a radiation intensity detected by the imaging detector. In order to ensure that the intensity of radiation rays detected by the reference detector is substantially similar to the intensity of radiation rays detected by the imaging detector, the thickness of the filter needs to be increased, or a filter including materials with low radiation transmittance needs to be used. However, using a thick filter may enlarge the size of the radiation measurement device, and using a filter including materials with low radiation transmittance may result in a difference between an energy spectrum detected by the reference detector and an energy spectrum detected by the imaging detector. Due to the above mentioned problems, conventional radiation measurement devices often occupy a large space and have a limited measurement accuracy.

An aspect of the present disclosure relates to a radiation measurement device. The radiation measurement device may comprise a scattering component and a first detector. The scattering component may be located between a radiation source of an imaging device and the first detector. The scattering component may be configured to scatter first radiation rays emitted by the radiation source into scattering rays. The first detector may be configured to collect first measurement data by detecting at least a portion of the scattering rays. An intensity of second radiation rays emitted by the radiation source toward a subject may be determined based on the first measurement data. Since an intensity of the scattering rays may be much smaller than an intensity of the original first radiation rays, the scattering component may only need to have a relatively small thickness to realize that an intensity of the at least a portion of the scattering rays detected by the first detector is substantially similar to an intensity of the second radiation rays detected by an imaging detector after passing though the subject. Compared with the conventional radiation measurement device, the radiation measurement device of the present disclosure may occupy a relatively small space. Moreover, in some embodiments, the scattering component and a filter configured to filter the second radiation rays may include the same material. In such cases, an energy spectrum of the detected scattering rays may be substantially the same as an energy spectrum of the second radiation rays detected by the imaging detector, thereby improving the accuracy of the obtained intensity of the second radiation rays.

In addition, the scattering component may be configured to transmit the first radiation rays into transmission rays. The first detector may be also configured to collect second measurement data by detecting at least a portion of the transmission rays. A focal point of the radiation source may be determined based on the second measurement data. That is, the radiation measurement device of the present disclosure may also realize the real-time measurement of the focal point of the radiation source.

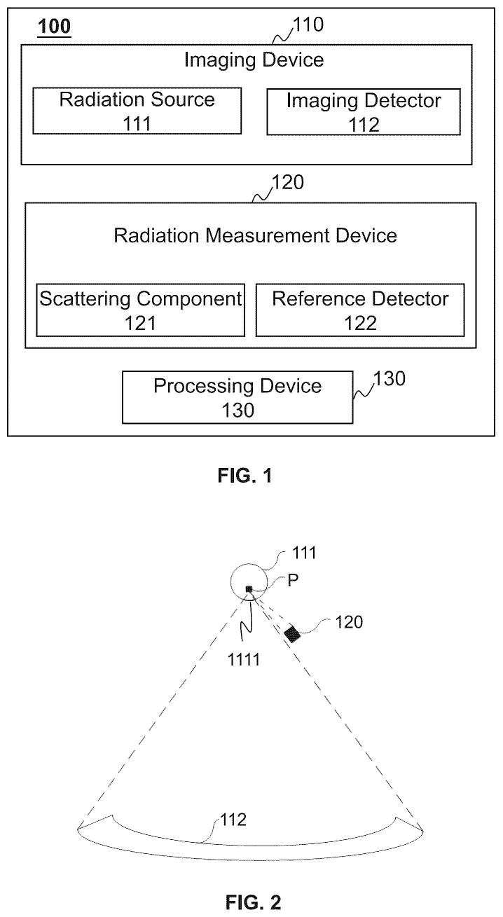

FIG. 1 is a block diagram illustrating an exemplary imaging system 100 according to some embodiments of the present disclosure. As shown in FIG. 1, the imaging system 100 may include an imaging device 110, a radiation measurement device 120, and a processing device 130.

The imaging device 110 may be configured to generate or provide image data related to a subject via scanning the subject. In some embodiments, the subject may include a biological subject and/or a non-biological subject. For example, the subject may include a specific portion of a body, such as a heart, a breast, or the like. In some embodiments, the imaging device 110 may be or include an X-ray imaging device, for example, a computed tomography (CT) scanner (e.g., a microCT scanner, an industrial CT scanner), a digital radiography (DR) scanner (e.g., a mobile digital radiography), a digital subtraction angiography (DSA) scanner, a dynamic spatial reconstruction (DSR) scanner, an X-ray microscopy scanner, a multimodality scanner, etc. In some embodiments, the X-ray imaging device may be, for example, a C-shape X-ray imaging device, an upright X-ray imaging device, a suspended X-ray imaging device, or the like.

In some embodiments, the imaging device 110 may include a radiation source 111 and an imaging detector 112 (also referred to as a second detector). During a scan, the radiation source 111 may be configured to generate and/or emit second radiation rays (or referred to as imaging radiation rays) (e.g., X-rays) toward the subject, and the imaging detector 112 may be configured to collect scan data by detecting the second radiation rays that pass through the subject. The second radiation rays may include a particle ray, a photon ray, or the like, or a combination thereof. In some embodiments, the second radiation rays may include radiation particles (e.g., neutrons, protons, electrons, p-mesons, heavy ions), radiation photons (e.g., X-ray, y-ray, ultraviolet, laser), or the like, or a combination thereof. In some embodiments, the imaging detector 112 may include a plurality of detecting units configured to collect the scan data. Exemplary detector units of the imaging detector 112 may include a scintillation detector (e.g., a cesium iodide detector), a gas detector, etc. The detecting units may be arranged in a single row or multiple rows.

The radiation measurement device 120 may be configured to collect measurement data reflecting the operation status of the radiation source 111. The operation status of the radiation source 111 may be defined by one or more operation parameters. Exemplary operation parameters may include an intensity of the second radiation rays emitted by the radiation source 111 (referred to as an intensity of the second radiation rays for brevity), a focal point of the radiation source 111, or the like.

In some embodiments, the radiation source 111 may be configured to emit first radiation rays (or referred to as reference radiation rays) toward the radiation measurement device 120 while emitting the second radiation rays. The radiation measurement device 120 may collect the measurement data based on the first radiation rays.

In some embodiments, the radiation measurement device 120 may include a scattering component 121 and a reference detector 122 (also referred to as a first detector). The scattering component 121 may be located between the radiation source 111 and the reference detector 122. In some embodiments, the scattering component 121 may be configured to scatter the first radiation rays emitted by the radiation source 111 into scattering rays. The reference detector 122 may be configured to collect first measurement data by detecting at least a portion of the scattering rays. The first measurement data may be used to determine an intensity of the second radiation rays. In some embodiments, the scattering component 121 may be configured to transmit the first radiation rays into transmission rays. The reference detector 122 may be configured to collect second measurement data by detecting at least a portion of the transmission rays. The second measurement data may be used to determine a focal point of the radiation source 111.

In some embodiments, the reference detector 122 may include one or more first detecting units configured to collect the first measurement data and/or one or more second detecting units configured to collect the second measurement data. In some embodiments, a detector unit of the reference detector 122 may include a scintillation detector (e.g., a cesium iodide detector), a gas detector, etc. The detecting units may be arranged in a single row or multiple rows. In some embodiments, the one or more first detecting units and the one or more second detecting units may be spaced apart from each other. A distance between the one or more first detecting units and the one or more second detecting units may be greater than a distance threshold. The distance threshold may be set according to an actual need, such as the size of the reference detector 122.

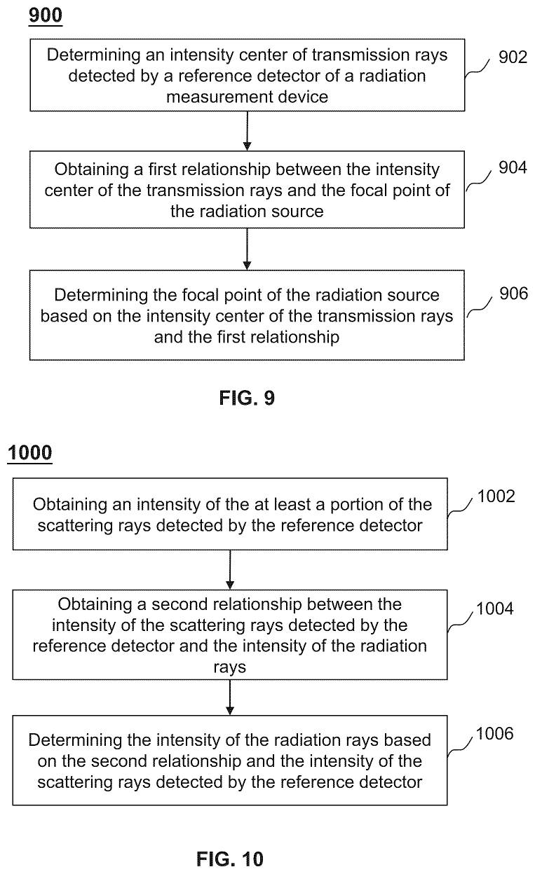

The processing device 130 may be configured to process data and/or information relating to one or more other components of the imaging system 100. In some embodiments, the processing device 130 may determine or analyze the operation status of the radiation source 111 based on measurement data collected by the radiation measurement device 120. For example, the processing device 130 may be configured to determine the focal point of the radiation source 111 based on the second measurement data. More descriptions for the determination of the focal point of the radiation source 111 may be found elsewhere in the present disclosure (e.g., FIG. 9 and the descriptions thereof). As another example, the processing device 130 may be configured to determine an intensity of the second radiation rays based on the first measurement data. The intensity of the second radiation rays may reflect the radiation intensity emitted by the radiation source 111 for scanning the subject. More descriptions for the determination of the intensity of the second radiation rays may be found elsewhere in the present disclosure (e.g., FIG. 10 and the descriptions thereof). As yet another example, the processing device 130 may be configured to obtain scan data of the subject collected by the imaging detector 130 during the scan of the subject. The processing device 130 may determine an intensity of radiation rays absorbed by the subject during the scan based on the scan data and the intensity of the second radiation rays. More descriptions for the determination of the intensity of radiation rays absorbed by the subject may be found elsewhere in the present disclosure (e.g., FIG. 10 and the descriptions thereof).

In some embodiments, methods disclosed in the present disclosure (e.g., the process 900, the process 1000, etc.) may be implemented in the imaging system 100. For example, the methods may be stored in a storage device as a form of instructions, and invoked and/or executed by the processing device 130 (e.g., one or more modules as illustrated in FIG. 8).

In some embodiments, the radiation measurement device 120 may be located outside a tube port of the radiation source 111. For example, FIG. 2 is a schematic illustrating an exemplary position of the radiation measurement device 120 according to some embodiments of the present disclosure. As shown in FIG. 2, the radiation source 111 includes a tube port 1111 for the second radiation rays to pass through to irradiate the subject. The radiation measurement device 120 may be located outside the tube port 1111 of the radiation source 111, so that the second radiation rays emitted toward the subject may not be blocked by the radiation measurement device 120. In some embodiments, a distance between the radiation measurement device 120 and the radiation source 111 may be smaller than a distance between the subject and the radiation source 111, so that the first radiation rays emitted toward the radiation measurement device 120 may not be blocked by the subject.

In some embodiments, the imaging system 100 may include one or more other components, such as a storage device, a terminal, etc. The storage device may store data, instructions, and/or any other information. In some embodiments, the storage device may store data obtained from one or more components (e.g., the imaging device 110, the radiation measurement device 120, the processing device 130, and/or the terminal, etc.) of the imaging system 100. In some embodiments, the storage device may store data and/or instructions that the processing device 130 may execute or use to perform exemplary methods described in the present disclosure.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart from the scope of the present disclosure. For example, the processing device 130 may be part of the radiation measurement device 120. As another example, the imaging system 100 in the present disclosure may also include multiple processing devices 130, and thus operations of a method that are performed by one processing device 130 as described in the present disclosure may be aslo jointly or separately performed by the multiple processing devices 130.

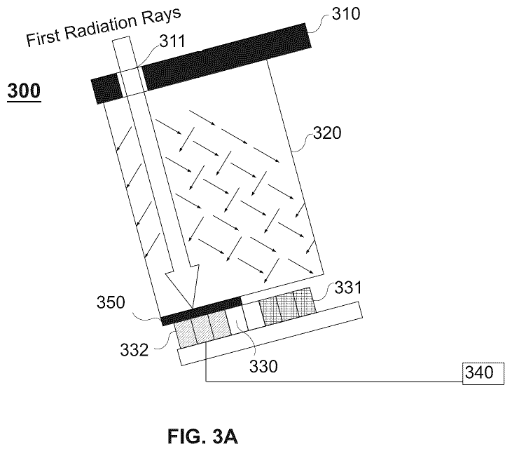

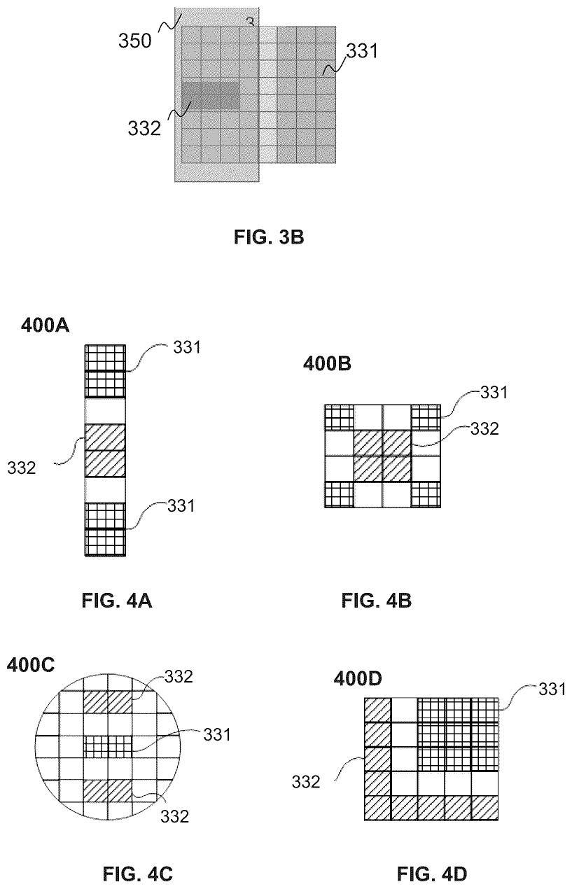

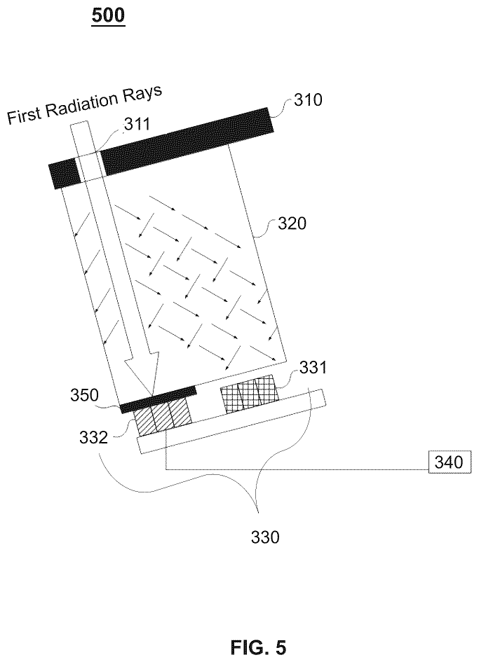

In some embodiments, the imaging system 100 may include one or more other components. For example, the imaging device 110 may include a filter located between the radiation source 111 and the imaging detector 112. As another example, the radiation measurement device 120 may include a blocking component disposed between the scattering component 121 and the radiation source 111, an attenuation component disposed between the scattering component 121 and the reference detector 122, etc. More descriptions regarding the filter, the blocking component, and the attenuation component may be found elsewhere in the present disclosure (e.g., FIG. 3A, FIGS. 5-7, and the descriptions thereof).

FIG. 3A is a schematic illustrating an exemplary radiation measurement device 300 according to some embodiments of the present disclosure. In some embodiments, the radiation measurement device 300 may be an exemplary embodiment of the radiation measurement device 120 of the imaging system 100 as described in FIG. 1. As shown in FIG. 3A, the radiation measurement device 300 may include a blocking component 310, a scattering component 320, and a reference detector 330.

The blocking component 310 may be located between a radiation source of an imaging device and the scattering component 320. The radiation source and the imaging device may be the same as or similar to the radiation source 111 and the imaging device 110, respectively, as described in FIG. 1. The blocking component 310 may include a through hole 311 for first radiation rays emitted by the radiation source to pass through to irradiate the scattering component 320. The blocking component 310 may include material that can block the transmission of radiation rays emitted by the radiation source, for example, tungsten, etc. A size of the blocking component 310 may be determined according to the scattering component 320. For example, a projected area of the blocking component 310 along a propagation direction of the first radiation rays may cover the scattering component 320. A thickness of the blocking component 310 along the propagation direction of the first radiation rays (indicated by the arrow in FIG. 3A) may be determined according to actual needs. In some embodiments, the blocking component 310 may include a tungsten plate having a certain thickness (e.g., 2 millimeters). For brevity, the thickness of a component along the propagation direction of the first radiation rays is referred to as the thickness.

The scattering component 320 may be located between the blocking component 310 and the reference detector 330. In some embodiments, the scattering component 320 may be configured to scatter the first radiation rays into scattering rays and/or transmit the first radiation rays into transmission rays. At least a portion of the scattering rays and/or at least a portion of the transmission rays may be used to monitor the operation status of the radiation source.

In some embodiments, as aforementioned, the radiation source may emit second radiation rays toward a subject to scan the subject, and a filter may be deposited between the radiation source and the subject to filter the second radiation rays. The scattering component 320 and the filter may include the same material, such as aluminum, Teflon, etc. In such cases, an energy spectrum of the detected scattering rays may be substantially the same as an energy spectrum of the second radiation rays detected by the imaging detector, thereby improving the accuracy of an intensity of the second radiation rays determined based on the scattering rays detected by scattering component 320.

In some embodiments, the scattering component 320 (e.g., the position, the shape, and/or the size of the scattering component 320) may be configured such that an intensity of the at least a portion of the scattering rays detected by the reference detector 330 (referred to as a first radiation intensity detected by the reference detector 330) may be substantially similar to an intensity of the second radiation rays detected by the imaging detector after passing though the subject (referred to as a second radiation intensity detected by the imaging detector) during the scan of the subject. The first radiation intensity detected by the reference detector 330 may be measured by, for example, a total radiation intensity, an average radiation intensity detected by the first detecting unit(s). The second radiation intensity detected by the imaging detector may be measured by, for example, a total radiation intensity, an average radiation intensity detected by imaging detecting unit(s) of the imaging detector. As used herein, if a difference between two intensities is within a difference threshold, the two intensities may be deemed as being substantially similar to each other. For example, the first radiation intensity may be 1-3 times the second radiation intensity.

Since an intensity of the scattering rays may be much smaller than an intensity of the original first radiation rays, the scattering component 320 may only need to have a relatively small thickness to realize that the first radiation intensity detected by the reference detector 330 is substantially similar to the second radiation intensity detected by the imaging detector during the scan of the subject. However, a reference detector of a conventional radiation measurement device is usually placed near the radiation source to collect measurement data by detecting radiation rays passing through a flitter. In order to reduce the radiation intensity detected by the reference detector, the filter needs to have a thickness much greater than the thickness of the scattering component, which results in that the conventional radiation measurement device occupies a large space. Compared with the conventional radiation measurement device, the radiation measurement device 300 may occupy a relatively small space.

In some embodiments, a thickness of the scattering component 320 may be determined according to the intensity of the second radiation rays and a distance between the scattering component 320 and the radiation source. For example, the farther the distance, the smaller the thickness may be, and the closer the distance, the greater the thickness may be.

The reference detector 330 may be configured to collect measurement data reflecting an operation status of the radiation source. The operation status of the radiation source may be defined by one or more operation parameters. Exemplary operation parameters may include an intensity of second radiation rays emitted toward a subject for scanning the subject, a focal point of the radiation source, or the like. In some embodiments, the reference detector 330 may collect first measurement data by detecting the at least a portion of the scattering rays. The first measurement data may be used to determine an intensity of the second radiation rays. Additionally or alternatively, the reference detector 330 may collect second measurement data by detecting the at least a portion of the transmission rays. The second measurement data may be used to determine a focal point of the radiation source.

In some embodiments, the reference detector 330 may include one or more first detecting units 331 configured to collect the first measurement data and/or one or more second detecting units 332 configured to collect the second measurement data. As shown in FIG. 3A, the one or more first detecting units 331 and the one or more second detecting units 332 may be located at a side of the scattering component 320 away from the blocking component 310 (i.e., the bottom of the scattering component 320 shown in FIG. 3A). A projected area of the scattering component 320 along the propagation direction of the first radiation rays may cover the one or more first detecting units 331 and the one or more second detecting units 332.

In some embodiments, a size of the through hole 311 may be determined according to a size of the one or more second detecting units 332. For example, the size of the through hole 311 may be equal to or larger than a pixel size corresponding to a second detecting unit 332. As another example, a projected area of the one or more second detecting units 332 along an opposite direction of the propagation direction of the first radiation rays may cover the through hole 311. In some embodiments, a position of the through hole 311 may be determined according to a position of the one or more second detecting units 332. For example, the through hole 311 and the one or more second detecting units 332 may be located on a same path of the propagation of the first radiation rays. As another example, an extension of a line connecting a focal point of the radiation source and the center of the through hole 311 may pass through the one or more second detecting units 332.

In some embodiments, the one or more first detecting units 331 and the one or more second detecting units 332 may be spaced apart from each other. A distance between the one or more first detecting units 331 and the one or more second detecting units 332 may be greater than a distance threshold. Merely by way of example, FIG. 3B illustrates a top view of the reference detector 330 according to some embodiments of the present disclosure. As shown in FIG. 3B, the reference detector 330 includes a first array of detecting units arranged in 8 rows and 8 columns. Each row of the first array includes 8 detecting units and each column of the first array includes 8 detecting units. The second detecting units 332 may include 6 detecting units located in the middle of the left 3 columns of the first array, and the first detecting units 331 may include the right 3 columns of detecting units of the first array. The first detecting units 331 and the second detecting units 332 are separated by two columns of detecting units. In this way, the first detecting units 331 may not detect the at least a portion of the transmission rays, thereby reducing or eliminating interference between the first detecting unit(s) 331 and the second detecting unit(s) 332, and improving the accuracy of the first measurement data and the second measurement data.

It should be understood that the reference detector 330 illustrated in FIG. 3B is provided for illustration purposes, and is not intended to be limiting. The one or more first detecting units 331 and the one or more second detecting units 332 may be arranged in any other form according to actual needs. For example, FIGS. 4A-4D illustrates top views of exemplary reference detectors according to some embodiments of the present disclosure. As shown in FIG. 4A, a reference detector 400A includes a second array of detecting units arranged in one column. The second array includes 8 detecting units. Four first detecting units 331 may be located at two ends of the second array, and two second detecting units 332 may be located at the middle of the second array. The first detecting units 331 and the second detecting units 332 are separated by one detecting unit.

As shown in FIG. 4B, a reference detector 400B includes a third array of detecting units arranged in 4 rows and 4 columns. The first detecting units 331 may be located at a periphery of the third array and the second detecting units 332 may be located at the middle of the third array. For example, as shown in FIG. 4B, each row of the third array includes 4 detecting units and each column of the third array includes 4 detecting units. Four first detecting units 331 may be located at four vertexes of the third array. Four second detecting units 332 are arranged as a 2*2 array and located in the middle of the third array. The first detecting units 331 and the second detecting units 332 are separated by one detecting unit.

As shown in FIG. 4C, the reference detector 400C includes a fourth array of detecting units arranged in a circle. The first detecting units 331 may be located at the middle of the fourth array and the second detecting units 332 may be located at a periphery of the fourth array. For example, as shown in FIG. 4C, two first detecting units 331 may be located at the middle of the fourth array. Fourth second detecting units 332 may be located at periphery of the fourth array. The first detecting units 331 and the second detecting units 332 are separated by one row of detecting units.

As shown in FIG. 4D, the reference detector 400D includes a fifth array of detecting units arranged in 5 rows and 5 columns. The first detecting units 331 may be located at the top right of the fifth array and the second detecting units 332 may be located at the left and/or bottom of the fifth array. For example, as shown in FIG. 4D, each row of the fifth array includes 5 detecting units and each column of the fifth array includes 5 detecting units. Nine first detecting units 331 may be located at the top right of the fifth array. Nine second detecting units 332 may be located in the left and bottom of the fifth array. The first detecting units 331 and the second detecting units 332 are separated by one row of detecting units and one column of detecting units.

In some embodiments, the radiation measurement device 300 may include or be connected to a processing device 340. The processing device 340 may be electrically connected to the reference detector 330. The processing device 340 may be configured to determine or analyze the operation status of the radiation source based on the measurement data collected by the reference detector 330. For example, the processing device 340 may be configured to determine the focal point of the radiation source based on the second measurement data. As another example, the processing device 340 may be configured to determine the intensity of the second radiation rays based on the first measurement data. As yet another example, the processing device 340 may be configured to obtain scan data of the subject collected by the imaging detector during the scan of the subject. The processing device 340 may determine an intensity of radiation rays absorbed by the subject during the scan based on the scan data and the intensity of the second radiation rays. More descriptions for the determination of the focal point of the radiation source, the intensity of the second radiation rays, and the intensity of radiation rays absorbed by the subject may be found elsewhere in the present disclosure (e.g., FIGS. 9 and 10, and the descriptions thereof). In some embodiments, the processing device 340 may be the same as or similar to the processing device 130 described in FIG. 1.

In some embodiments, an intensity of the transmission rays transmitted from the scattering component 320 may be relatively high, which may exceed a radiation detection range of the reference detector 330 and damage the reference detector 330, thereby affecting the second measurement data collected by the one or more second detecting units. In order to avoid this situation, an attenuation component 350 may be disposed between the scattering component 320 and the reference detector 330. The attenuation component 350 may be configured to attenuate the transmission rays transmitted from the scattering component 320, so that the intensity of the transmission rays detected by the reference detector 330 may be reduced to the desired level. The material of the attenuation component 350 may include tungsten, aluminum, lead, tungsten alloy, and/or other materials, etc.

In some embodiments, a size of the attenuation component 350 may be determined based on the intensity of the transmission rays passing through the scattering component 320, a desired intensity of the transmission rays detected by the second detecting units 332, a size of the one or more second detecting units 332, etc. For example, a thickness of the attenuation component 350 may be determined based on the intensity of the transmission rays and the desired intensity of the portion of the transmission rays that passes through the attenuation component 350. As another example, a projected area of the attenuation component 350 along the propagation direction of the first radiation rays may cover the one or more second detecting units 332.

FIG. 5 is a schematic illustrating an exemplary radiation measurement device 500 according to some embodiments of the present disclosure. In some embodiments, the radiation measurement device 500 may be an exemplary embodiment of the radiation measurement device 120 of the imaging system 100 as described in FIG. 1. As shown in FIG. 5, the radiation measurement device 500 may be similar to the radiation measurement device 300 as described in FIG. 3A, except that there are no other detecting units between the one or more first detecting units 331 and the one or more second detecting units 332.

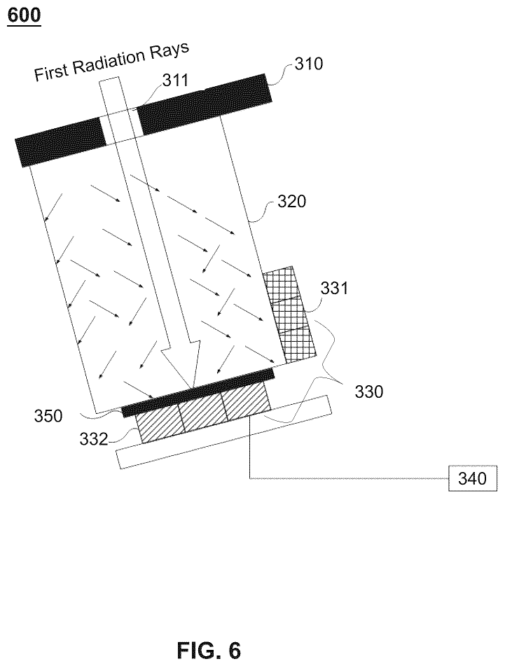

FIG. 6 is a schematic illustrating an exemplary radiation measurement device 600 according to some embodiments of the present disclosure. In some embodiments, the radiation measurement device 600 may be an exemplary embodiment of the radiation measurement device 120 of the imaging system 100 as described in FIG. 1. As shown in FIG. 6, the radiation measurement device 600 may be similar to the radiation measurement device 300 as described in FIG. 3A, except that the one or more first detecting units 331 are located on the right side of the scattering component 320, and the one or more second detecting units 332 are located on the bottom side of the scattering component 320.

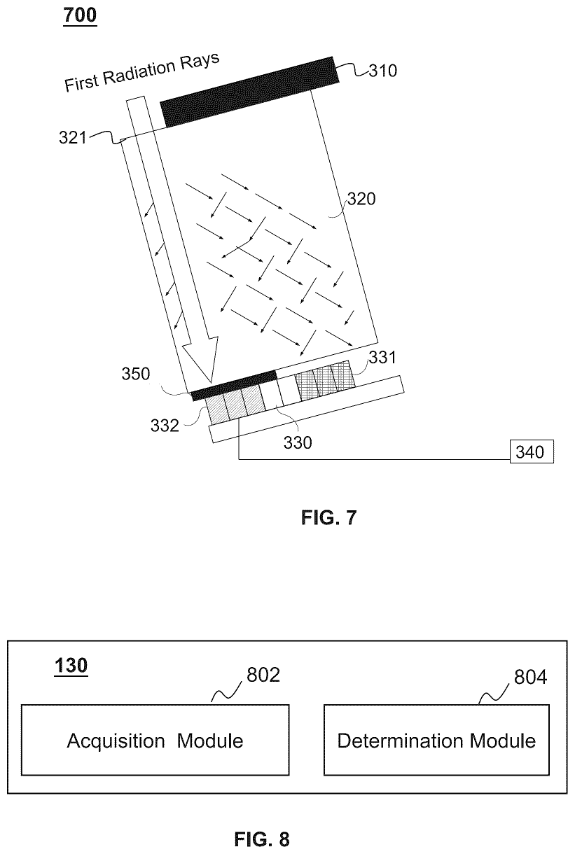

FIG. 7 is a schematic illustrating an exemplary radiation measurement device 700 according to some embodiments of the present disclosure. In some embodiments, the radiation measurement device 700 may be an exemplary embodiment of the radiation measurement device 120 of the imaging system 100 described in FIG. 1. As shown in FIG. 7, the radiation measurement device 700 may be similar to the radiation measurement device 300 as described in FIG. 3A, except that the blocking component 310 only covers a portion of the top surface of the scattering component 320, and the first radiation rays are transmitted into the scattering component 320 through a remaining portion 321 of the scattering component 320 that is not covered by the blocking component 310.

FIG. 8 is a block diagram illustrating an exemplary processing device 130 according to some embodiments of the present disclosure. As shown in FIG. 8, the processing device 130 may include an acquisition module 802 and a determination module 804.

The acquisition module 802 may be configured to obtain information relating to the imaging system 100. For example, the acquisition module 802 may obtain a first relationship between the intensity center of the detected transmission rays and the focal point of the radiation source. More descriptions regarding the obtaining of the first relationship between the intensity center of the detected transmission rays and the focal point of the radiation source may be found elsewhere in the present disclosure. See, e.g., operation 904 in FIG. 9, and relevant descriptions thereof. As another example, the acquisition module 802 may obtain an intensity of the at least a portion of the scattering rays detected by the reference detector (referred to as a first radiation intensity detected by the reference detector). More descriptions regarding the obtaining of the intensity of the at least a portion of the scattering rays detected by the reference detector may be found elsewhere in the present disclosure. See, e.g., operation 1002 in FIG. 10, and relevant descriptions thereof. As still another example, the acquisition module 802 may obtain a second relationship between the intensity of the detected scattering rays and the intensity of the second radiation rays. More descriptions regarding the obtaining of the second relationship may be found elsewhere in the present disclosure. See, e.g., operation 1004 in FIG. 10, and relevant descriptions thereof.

The determination module 804 may be configured to determine an intensity center of transmission rays detected by a reference detector of a radiation measurement device. More descriptions regarding the determining of the intensity center of transmission rays detected by a reference detector of a radiation measurement device may be found elsewhere in the present disclosure. See, e.g., operation 902 in FIG. 9, and relevant descriptions thereof.

In some embodiments, the determination module 804 may be further configured to determine the focal point of the radiation source based on the intensity center of the detected transmission rays and the first relationship. More descriptions regarding the determining of the focal point of the radiation source may be found elsewhere in the present disclosure. See, e.g., operation 906 in FIG. 9, and relevant descriptions thereof.

In some embodiments, the determination module 804 may be configured to determine the intensity of the second radiation rays based on the second relationship and the intensity of the scattering rays detected by the reference detector. More descriptions regarding the determining of the intensity of the second radiation rays may be found elsewhere in the present disclosure. See, e.g., operation 1006 in FIG. 10, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the processing device 130 may further include a storage module (not shown in FIG. 8). The storage module may be configured to store data generated during any process performed by any component of the processing device 130. As another example, each of at least some components of the processing device 130 may include a storage apparatus. Additionally or alternatively, at least some components of the processing device 130 may share a common storage apparatus.

FIG. 9 is a flowchart illustrating an exemplary process 900 for determining a focal point of a radiation source of an imaging device according to some embodiments of the present disclosure.

In 902, the processing device 130 (e.g., the determination module 804) may determine an intensity center of transmission rays detected by a reference detector of a radiation measurement device.

In some embodiments, the radiation measurement device may be any one of the radiation measurement device 120, the radiation measurement device 300, the radiation measurement device 500, the radiation measurement device 600, the radiation measurement device 700 as described in FIGS. 1-7.

As described elsewhere in this disclosure (e.g., FIGS. 1-7 and the relevant descriptions), a radiation source may emit first radiation rays toward the radiation measurement device, the first radiation rays may be transmitted through a scatter component of the radiation measurement device, and at least a portion of transmission rays passing through the scattering component may be detected by the reference detector. One or more second detecting units of the reference detector may be configured to collect second measurement data by detecting the at least a portion of the transmission rays. The second measurement data may include a radiation intensity of transmission rays detected by each of the second detecting unit(s). For brevity, the transmission rays detected by the second detecting unit(s) may be referred to as detected transmission rays.

In some embodiments, the processing device 130 may determine the intensity center of the detected transmission rays based on the second measurement data. As used herein, the intensity center of the detected transmission rays may be regarded as the center of mass of the detected transmission rays. It may be assumed that the radiation intensities of the detected transmission rays are concentrated at a point (i.e., the intensity center). In some embodiments, the processing device 130 may determine a distribution of the radiation intensities detected by the second detecting unit(s). For example, the processing device 130 may generate a radiation intensity distribution map based on the radiation intensities detected by the second detecting unit(s). The processing device 130 may further determine the intensity center of the detected transmission rays based on the radiation intensity detected by each second detecting unit and the radiation intensity distribution map. For example, the processing device 130 may determine the intensity center of the detected transmission rays using an algorithm for determining a center of mass. Merely for illustration purposes, the processing device 130 may determine the intensity center of the detected transmission rays according to Equation (1) as below:

$$\overline{x} = \frac{{\sum }_{i = 1}^{n} {x}_{{i}^{*}} {I}_{i}}{{\sum }_{i = 1}^{n} {I}_{i}} , \qquad ( 1 )$$

where x̅ denotes the intensity center of the detected transmission rays, n denotes a count of the second detecting unit(s), xᵢ denotes a position (e.g., a serial number) of the ith second detecting unit, and Iᵢ denotes the radiation intensity detected by the ith second detecting unit.

In some embodiments, the processing device 130 may determine the intensity center of the detected transmission rays based on a radiation intensity difference distribution map. Each value in the radiation intensity difference distribution map may be a complement number of one radiation intensity in the radiation intensity distribution map. That is, a peak in the radiation intensity distribution map may correspond to a trough in the radiation intensity difference distribution map, and a trough in the radiation intensity distribution map may correspond to a peak in the radiation intensity difference distribution map. In some embodiments, the processing device 130 may determine an intensity characteristic value of the detected transmission rays according to the radiation intensities detected by the second detecting unit(s). The intensity characteristic value may be, for example, an average value or a maximum value of the radiation intensities detected by the second detecting unit(s). Selecting the average value as the intensity characteristic value may reduce or eliminate the effect of abnormal radiation intensities detected by the second detecting unit(s) due to electrical signal noises or interferences, thereby improving the accuracy of the determined intensity center. The processing device 130 may determine a difference between the intensity characteristic value and each of the radiation intensities in the radiation intensity distribution map, so as to obtain the radiation intensity difference distribution map.

In some embodiments, the processing device 130 may perform a correct operation on the radiation intensity difference distribution map. For example, in response to determining that a difference between the intensity characteristic value and a radiation intensity in the radiation intensity distribution map is smaller than 0, the processing device 130 may update the difference between the intensity characteristic value and the radiation intensity to 0. In response to determining that the difference between the intensity characteristic value and an intensity in the radiation intensity distribution map is not smaller than 0, the processing device 130 may not modify the radiation intensity difference map. The processing device 130 may determine the difference distribution according to the updated differences. In this way, the accuracy of the determined intensity center may be improved.

In 904, the processing device 130 (e.g., the acquisition module 802) may obtain a first relationship between the intensity center of the detected transmission rays and the focal point of the radiation source.

Normally, during a scan performed by the imaging device, the focal point of the radiation source may move and the radiation dose emitted by the radiation source may fluctuate. Assuming that the focal point of the radiation source remains substantially unchanged and an intensity of the first radiation rays changes, the radiation intensities detected by the second detection unit(s) may be proportionally enlarged or reduced, that is, the intensity center of the detected transmission rays may remain unchanged but the total radiation intensity detected by the second detecting unit(s) may be proportionally enlarged or reduced. Assuming that the intensity of the first radiation rays remains substantially unchanged and the focal point of the radiation source changes, the radiation intensities detected by the second detection unit(s) may only be related to the movement of the focal point of the radiation source. In such cases, a unique corresponding relationship (i.e., the first relationship) may exist between the intensity center of the detected transmission rays and the focal point of the radiation source. The focal point of the radiation source may be determined based on the intensity center and the first relationship.

In some embodiments, the radiation source may be directed to perform a plurality of reference scans (also referred to as first reference scans) on the air. During each of the plurality of reference scans, the radiation source may emit reference first radiation rays toward the reference detector and reference second radiation rays toward a calibration detector (e.g., the imaging detector described in the FIGS. 1-7). For each of the plurality of reference scans, the processing device 130 may determine a reference intensity center of transmission rays detected by the reference detector and a reference focal point of the radiation source during the reference scan. In some embodiments, the determination of the reference intensity center may be performed in a similar manner as that of the intensity center as described in operation 902, and the descriptions of which are not repeated here. The reference focal point of the radiation source may be determined based on one or more parameters of the calibration detector and the reference second radiation rays detected by the calibration detector. Exemplary parameters of the calibration detector may include a shape, a size, a position, etc. of the calibration detector. For example, the reference focal point of the radiation source may be determined according to the shape, the size, and the position of the calibration detector, a propagation direction of the reference second radiation rays.

In some embodiments, during each of the plurality of reference scans, the radiation source may only emit the reference first radiation rays toward the reference detector. The reference focal point of the radiation source may be determined according to one or more parameters (e.g., a shape, a size, a position, of the reference detector, etc.) of the reference detector and the reference first radiation rays. For example, referring back to FIG. 2, a reference focal point P of the radiation source 111 may be determined according to one or more parameters of the imaging detector 112 or the reference detector 122 of the radiation measurement device 120. In some embodiments, because a size of the calibration detector may be larger than a size of the reference detector, the reference focal point determined based on the calibration detector may be more accurate than the reference focal point determined with the reference detector.

The processing device 130 may determine the first relationship based on the reference intensity centers and the reference focal points of the plurality of reference scans. In some embodiments, the first relationship may be represented by, for example, a table, a formula, or a model. Merely by way of example, the processing device 130 may perform a fitting on the reference intensity centers and the reference focal points of the plurality of reference scans to obtain a function or a model, and the obtained function or the model may be determined as the first relationship.

In some embodiments, the first relationship may be previously generated and stored in a storage device (e.g., the storage device of the imaging system 100, or an external source). The processing device 130 may retrieve the first relationship directly from the storage device.

In 906, the processing device 130 (e.g., the determination module 804) may determine the focal point of the radiation source based on the intensity center of the detected transmission rays and the first relationship.

For example, if the first relationship is represented as a formula, the processing device 130 may determine the focal point of the radiation source by inputting the intensity center of the detected transmission rays into the formula. As another example, if the first relationship is represented as a table, the processing device 130 may determine the focal point of the radiation source by looking up the table based on the intensity center of the detected transmission rays.

In some embodiments, the processing device 130 may determine the intensity center of the detected transmission rays continuously or intermittently (e.g., periodically) during a scan of a subject performed by the imaging device, and further determine the focal point of the radiation source based on the determined intensity center of the detected transmission rays, so that the change of the focal point of the radiation source over time may be tracked.

FIG. 10 is a flowchart illustrating an exemplary process 1000 for determining an intensity of second radiation rays according to some embodiments of the present disclosure.

The second radiation rays may be emitted by a radiation resource of an imaging device toward a subject for scanning the subject. As used herein, the subject may include a biological subject and/or a non-biological subject. For example, the subject may be a human being, an animal, or a portion thereof. As another example, the subject may be a phantom. In some embodiments, the subject may be a patient, or a portion of the patient (e.g., the chest, the breast, and/or the abdomen of the patient).

The intensity of the second radiation rays may be determined using a radiation measurement device. In some embodiments, the radiation measurement device may be any one of the radiation measurement device 120, the radiation measurement device 300, the radiation measurement device 500, the radiation measurement device 600, the radiation measurement device 700 described in FIGS. 1-7.

As described elsewhere in this disclosure (e.g., FIGS. 1-7 and the relevant descriptions), the radiation source may emit first radiation rays toward the radiation measurement device, the first radiation rays may be scattered by a scatter component of the radiation measurement device into scattering rays, and at least a portion of scattering rays passing through the scattering component may be detected by the reference detector. One or more first detecting units of the reference detector may be configured to collect first measurement data by detecting the at least a portion of the scattering rays. The first measurement data may include a radiation intensity of scattering rays detected by each of the first detecting unit(s). For brevity, the scattering rays detected by the first detecting unit(s) may be referred to as detected scattering rays.

In 1002, the processing device 130 (e.g., the acquisition module 802) may obtain an intensity of the at least a portion of the scattering rays detected by the reference detector (referred to as a first radiation intensity detected by the reference detector).

The first radiation intensity may be represented by, for example, an average radiation intensity detected by the first detecting unit(s), a total radiation intensity detected by the first detecting unit(s), a distribution of the radiation intensities detected by the first detecting unit(s), or the like, or any combination thereof.

In 1004, the processing device 130 (e.g., the acquisition module 802) may obtain a second relationship between the intensity of the detected scattering rays and the intensity of the second radiation rays.

In some embodiments, the radiation source may be directed to perform a plurality of second reference scans on the air. During each of the plurality of second reference scans, the radiation source may emit reference third radiation rays toward the radiation measurement device and reference fourth radiation rays toward an imaging detector (e.g., the imaging detector 112 described in FIG. 1) of the imaging device. For each of the plurality of second reference scans, the processing device 130 may obtain a first reference intensity measured by the reference detector and a second reference intensity measured by the imaging detector during the second reference scan.

In some embodiments, the obtaining of the first reference intensity may be performed in a similar manner as that of the first radiation intensity described in operation 1002, and the descriptions of which are not repeated here. In some embodiments, the imaging detector may include a plurality of imaging detecting units configured to detect the reference fourth radiation rays. The second reference intensity may be represented by, for example, a radiation intensity detected by each imaging detecting unit during the second reference scan, a sum of the radiation intensities detected by the imaging detecting units, a distribution of the radiation intensities detected by the imaging detecting units, or the like, or any combination thereof.

The processing device 130 may determine the second relationship based on the first reference intensities and the second reference intensities of the plurality of second reference scans. In some embodiments, the second relationship may be represented by, for example, a table, a formula, or a model. Merely by way of example, for each of the plurality of second reference scans, the processing device 130 may determine a ratio of the second reference intensity to the first reference intensity. The processing device 130 may determine a table (also referred to as an air calibration table) for representing the second relationship based on the plurality of ratios. For example, the second reference intensity may be represented by a distribution of the radiation intensities detected by the imaging detecting units, and the processing device 130 may determine an element in the air calibration table according to Equation (2) as below:

$$Ref A ( i , j ) \frac{I 0 ( i , j )}{I\_RD 0} , \qquad ( 2 )$$

where I0(i,j) denotes a radiation intensity detected by one imaging detecting unit at row i and column j in the imaging detector, I_RD0 denotes the first reference intensity, and RefA(i,j) denotes an element in the air calibration table corresponding to the imaging detecting unit at row i and column j.

In some embodiments, the second relationship may be previously generated and stored in a storage device (e.g., the storage device of the imaging system 100, or an external source). The processing device 130 may retrieve the second relationship directly from the storage device.

In 1006, the processing device 130 (e.g., the determination module 804) may determine the intensity of the second radiation rays based on the second relationship and the intensity of the scattering rays detected by the reference detector.

For example, the intensity of the second radiation rays may be represented by a radiation intensity emitted toward each imaging detecting unit, which may be determined according to Equation (3) as below:

I(i,j)=I_RD*RefA(i,j), (3)

where I(i,j) denotes a radiation intensity emitted toward an imaging detecting unit at row i and column j, I_RD denotes the intensity of the detected scattering rays, RefA(i,j) denotes an element in the air calibration table corresponding to the imaging detecting unit at row i and column j.

In some embodiments, the processing device 130 may be configured to obtain scan data of the subject collected by the imaging detector during the scan of the subject. The processing device 130 may determine an intensity of radiation rays absorbed by the subject during the scan based on the scan data and the intensity of the second radiation rays. Merely by way of example, the scan data of the subject may include a radiation intensity detected by each imaging detecting unit during the scan of the subject, and the processing device 130 may determine the intensity of radiation rays absorbed by the subject during the scan according to Equation (4) as below:

I_Abs(i,j)=I(i,j)−I_AfterObj(i,j), (4)

where I_Abs(i,j) denotes a radiation intensity absorbed by a region of the subject corresponding to an imaging detecting unit at row i and column j, I(i,j) denotes a radiation intensity emitted toward the imaging detecting unit at row i and column j, and I_AfterObj(i,j) denotes a radiation intensity detected by imaging detecting unit at row i and column j.

In some embodiments, an image of the subject may be generated based on the obtained intensity of radiation rays absorbed by the subject during the scan. For example, the processing device 130 may reconstruct the image of the subject based on the obtained intensity of radiation rays absorbed by the subject during the scan using an image reconstruction technique. Exemplary reconstruction techniques may include a 2-dimensional Fourier transform technique, a back projection technique (e.g., a convolution back projection technique, a filtered back projection technique), an iteration technique, etc. Exemplary iteration techniques may include an algebraic reconstruction technique (ART), a simultaneous iterative reconstruction technique (SIRT), a simultaneous algebraic reconstruction technique (SART), an adaptive statistical iterative reconstruction (ASIR) technique, a model-based iterative reconstruction (MBIR) technique, a sinogram affirmed iterative reconstruction (SAFIR) technique, or the like, or any combination thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including a subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may be aslo implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

1. A radiation measurement device, wherein
the radiation measurement device comprises a scattering component and a first detector,
the scattering component is located between a radiation source of an imaging device and the first detector, and configured to scatter first radiation rays emitted by the radiation source into scattering rays; and
the first detector is configured to collect first measurement data by detecting at least a portion of the scattering rays, the first measurement data reflecting an operation status of the radiation source.

2. The radiation measurement device of claim 1, wherein
the scattering component is further configured to transmit the first radiation rays into transmission rays; and
the first detector is further configured to collect second measurement data by detecting at least a portion of the transmission rays, the second measurement data reflecting the operation status of the radiation source.

3. The radiation measurement device of claim 2, wherein the first detector includes one or more first detecting units configured to collect the first measurement data and one or more second detecting units configured to collect the second measurement data.

4. The radiation measurement device of claim 3, wherein the one or more first detecting units and the one or more second detecting units are spaced apart from each other.

5. The radiation measurement device of claim 2, wherein the radiation measurement device further includes a processing device configured to determine, based on the second measurement data, a focal point of the radiation source.

6. The radiation measurement device of claim 5, wherein to determine, based on the second measurement data, a focal point of the radiation source, the processing device is configured to:
determine, based on the second measurement data, an intensity center of the at least a portion of the transmission rays detected by the first detector;
obtain a first relationship between the intensity center of the at least a portion of the transmission rays and the focal point of the radiation source; and
determine, based on the intensity center of the at least a portion of the transmission rays and the first relationship, the focal point of the radiation source.

7. The radiation measurement device of claim 2, wherein the radiation measurement device further includes an attenuation component disposed between the scattering component and the first detector, and the attenuation component is configured to attenuate the transmission rays transmitted from the scattering component.

8. The radiation measurement device of claim 1, wherein
the radiation source is further configured to scan a subject by emitting second radiation rays toward the subject;
the first measurement data includes an intensity of the at least a portion of the scattering rays detected by the first detector; and
the radiation measurement device further includes a processing device configured to determine, based on the intensity of the at least a portion of the scattering rays detected by the first detector, an intensity of the second radiation rays.

9. The radiation measurement device of claim 8, wherein to determine, based on the intensity of the at least a portion of the scattering rays detected by the first detector, an intensity of the second radiation rays, the processing device is configured to:
obtain a second relationship between the intensity of the at least a portion of the scattering rays detected by the first detector and the intensity of the second radiation rays; and
determine, based on the second relationship and the intensity of the at least a portion of the scattering rays detected by the first detector, the intensity of the second radiation rays.

10. The radiation measurement device of claim 8, wherein the processing device is further configured to:
obtain scan data of the subject collected by a second detector during the scan of the subject; and
determine, based on the scan data and the intensity of the second radiation rays, an intensity of radiation rays absorbed by the subject during the scan.

11. The radiation measurement device of claim 10, wherein
the imaging device further includes a filter located between the radiation source and the second detector, and configured to filter the second radiation rays; and
the scattering component and the filter include the same material.

12. The radiation measurement device of claim 1, wherein
the radiation source is further configured to scan a subject by emitting second radiation rays toward the subject;
the imaging device further includes a second detector configured to detect the second radiation rays after passing though the subject; and
the scattering component is configured such that an intensity of the at least a portion of the scattering rays detected by the first detector is substantially similar to an intensity of the second radiation rays detected by the second detector.

13. The radiation measurement device of claim 1, wherein
the radiation measurement device further includes a blocking component disposed between the scattering component and the radiation source, and
the blocking component includes a through hole for the first radiation rays to pass through to irradiate the scattering component.

14. A radiation measurement device, wherein
the radiation measurement device comprises a scattering component and a first detector,
the scattering component is located between a radiation source of an imaging device and the first detector, and configured to transmit first radiation rays emitted by the radiation source into transmission rays; and
the first detector is configured to collect first measurement data by detecting at least a portion of the transmission rays, the first measurement data reflecting an operation status of the radiation source.

15. (canceled)

16. The radiation measurement device of claim 14, wherein
the radiation source is further configured to scan a subject by emitting second radiation rays toward the subject;
the first measurement data includes an intensity of the at least a portion of the scattering rays detected by the first detector; and
the radiation measurement device further includes a processing device configured to determine, based on the intensity of the at least a portion of the scattering rays detected by the first detector, an intensity of the second radiation rays.

17. The radiation measurement device of claim 16, wherein to determine, based on the intensity of the at least a portion of the scattering rays detected by the first detector, an intensity of the second radiation rays, the processing device is configured to:
obtain a second relationship between the intensity of the at least a portion of the scattering rays detected by the first detector and the intensity of the second radiation rays; and
determine, based on the second relationship and the intensity of the at least a portion of the scattering rays detected by the first detector, the intensity of the second radiation rays.

18-20. (canceled)

21. The radiation measurement device of claim 14, wherein
the radiation measurement device further includes a blocking component disposed between the scattering component and the radiation source, and
the blocking component includes a through hole for the first radiation rays to pass through to irradiate the scattering component.

22. An imaging system, comprising a radiation source, a radiation measurement device, and a processing device, wherein
the radiation source is configured to emit first radiation rays toward the radiation measurement device and second radiation rays toward a subject;
the radiation measurement device includes a first detector and a scattering component located between the radiation source and the first detector, the scattering component being configured to scatter the first radiation rays into scattering rays, and the first detector being configured to collect first measurement data by detecting at least a portion of the scattering rays; and
the processing device is configured to determine, based on the first measurement data, feature information of the second radiation rays.

23. The radiation system of claim 22, wherein the radiation measurement device is located outside a tube port of the radiation source.

24. The radiation system of claim 22, wherein
the scattering component is further configured to transmit the first radiation rays into transmission rays;
the first detector is further configured to collect second measurement data by detecting at least a portion of the transmission rays; and
the processing device is further configured to determine, based on the second measurement data, second feature information of the second radiation rays.
